(19) United States
(12) Patent Application Publication
(10) Pub. No.: US 2012/0034629 A1
(43) Pub. Date: Feb. 9, 2012

(54) PREDICTION OF NON-FATAL AND FATAL ATHEROTHROMBOTIC EVENTS

(75) Inventors: Mathias Gehrmann, Leverkusen (DE); Johannes-Peter Stasch, Solingen (DE)

(73) Assignee: Siemens Healthcare Diagnostics Inc., Tarrytown, NY (US)

(21) Appl. No.: 12/673,780

(22) PCT Filed: Aug. 17, 2007
(86) PCT No.: PCT/EP07/07286

§ 371 Date: Aug. 23, 2011

(51) Int. Cl. G01N 33/566; C12M 1/34

(52) U.S. Cl. 435/7.92; 435/287.2

(57) ABSTRACT

The present invention relates to methods and systems for the prediction of atherothrombotic events in human subjects. Preferably the human subjects are afflicted with a cardiovascular disease, such as end-stage renal disease. Methods and systems of the invention are particularly suited to predict atherothrombotic events in patients on hemodialysis.

### BACKGROUND OF THE INVENTION

Cardiovascular mortality is remarkably high in patients on hemodialysis. Soluble CD154 (sCD154), a protein belonging to the TNF receptor superfamily, has been implicated in the pathogenesis of atheromatous plaque destabilization and thrombotic events.

Although hemodialysis techniques have improved exceptionally in the past decades, high mortality of patients on hemodialysis represents a major yet unsolved clinical issue. Despite a well accepted, albeit complex set of guidelines that have been introduced in clinical practice in the United States and in Europe, outcomes remain poor (1-3). Cardiovascular disease (sudden cardiac death, heart failure and atherothrombotic diseases) is the most common cause of death followed by infectious diseases (4-6). Chronic vascular inflammation seems to play a pivotal role in the pathogenesis of cardiovascular morbidity and mortality in end stage renal disease patients on hemodialysis. Particularly atherothrombotic diseases (myocardial infarction, stroke, mesenteric ischemia) are related to an activated vascular immune system in patients with impaired renal function (7-9). Recently, the CD40-CD154 system has been implicated in the pathogenesis of plaque destabilization leading to acute fatal and non-fatal myocardial infarctions and stroke (10;11). Studies in CD154 knockout mice suggest a similar impact of CD154 for the pathogenesis of mesenteric ischemic events (12). The CD40-CD154 system represents an important system that amplifies the endothelial cell responses to inflammation. CD40, a member of the TNF receptor superfamily, is constitutively expressed on platelets and endothelial cells. CD154 (named CD40 ligand or CD40L in the past), the ligand for CD40, is found on cells of the immune system (e.g. T lymphocytes) and on activated platelets. CD154 also exists in a soluble form (sCD154), which is shed from lymphocytes and platelets after cell activation. Interaction of CD154 with CD40 on endothelial cells results in increased expression of adhesion molecules such as E-selectin, ICAM-1, and VCAM-1, increased secretion of the chemokines interleukin-8, interleukin-6, and monocyte chemoattractant protein-1, and enhanced production of reactive oxygen species (10;11). Elevated levels of soluble and membrane-bound CD154 have been reported in patients with angina, and after cardiopulmonary bypass surgery (10;11;13;14). Moreover, in patients with unstable coronary artery disease, elevated sCD154 levels indicate an increased risk of cardiovascular events in a 6 months follow-up period (15).

It is known that the inflammatory marker C-reactive protein (CrP) predicts all-cause mortality (7;8;23). However, CrP failed to predict mortality and hospitalization due to atherothrombotic events (Table 4).

### SUMMARY OF THE INVENTION

The present invention relates to methods and systems for the prediction of atherothrombotic events in human subjects. Preferably the human subjects are afflicted with a cardiovascular disease, such as end-stage renal disease. Methods and systems of the invention are particularly suited to predict atherothrombotic events in patients on hemodialysis.

### BRIEF DESCRIPTION OF THE FIGURES

FIG. 1 depicts Kaplan-Meier curve analyses on the basis of CD154 plasma levels, for sudden cardiac death, death due to heart failure and infectious diseases. and the combined endpoint mortality and hospitalization for sudden cardiac death/arrhythmias, heart failure and infectious diseases. Solid lines, patients with CD154 levels of <6.42 ng/ml; dotted lines, patients with CD154 levels of >6.42 ng/ml. Survival rates were compared with the log rank test.

FIG. 2 depicts Kaplan-Meier curve analyses, on the basis of CD154 plasma levels, for fatal atherothrombotic events (myocardial infarction, stroke, mesenteric ischemia) and the combined endpoint mortality and hospitalization for atherothrombotic events (myocardial infarction, stroke, mesenteric ischemia) among patients with ESRD. Solid lines, patients with CD154 levels of <6.42 ng/ml; dotted lines, patients with CD154 levels of >6.42 ng/ml. Survival rates were compared with the log rank test.

### DETAILED DESCRIPTION OF THE INVENTION

An “atherothrombotic event” within the meaning of the invention shall have the ordinary meaning as understood by the person skilled in the art. An atherothrombotic event is characterized by a sudden plaque disruption leading to platelet activation and thrombus formation. Common atherothrombotic events are or can lead to myocardial infarction, stroke, mesenteric ischemia, ischemia, and/or peripheral arterial disease.

“CD154”, within the meaning of the invention, shall be understood as relating to the CD154 gene or to the protein encoded by this gene. CD154 has previously also been referred to as “CD40 ligand”, or “CD40L”. “sCD154” is short for “soluble CD154”.

The “determination of the level of CD154” shall be understood as being any determination of the concentration or of the relative amount of CD154 in a subject or in a sample from a subject. The determination of the level of CD154 is preferably performed in vitro. The determination of the level of CD154 is understood relate either to the amount of CD154 protein present in a subject or a sample or to the amount of nucleic acid coding for the CD154 protein in a subject or a sample.

“Fatal atherothrombotic events”, according to the invention are atherothrombotic events which lead to the death of the subject having such a fatal atherothrombotic event.

“Non-fatal atherothrombotic events”, within the meaning of the invention, shall be understood as being atherothrombotic events which do not lead to the death of the subject.

The expressions “gene chip” and “protein chip”, within the meaning of the invention, shall have the ordinary meaning of the term as understood by the person skilled in the art. Any planar arrangement of features on a solid support, on which features nucleic acids or proteins, respectively, can specifically bind and be detected, shall be considered to be a “gene chip” or “protein chip” according to the invention.

A “predetermined reference value”, within the meaning of the invention, shall be understood as being any value that is known prior to the determination of the test value which is to be compared to said predetermined reference value. Predetermined reference values are preferably stored in the memory of the device that performs the comparison of the test value with the predetermined reference value.

“Predicting an atherothrombotic event”, within the meaning of the invention, shall relate to the prediction whether or not an atherothrombotic event will be experienced by a subject. Alternatively, “predicting an atherothrombotic event” relates to the determination of the likelihood, that an atherothrombotic event will be experienced.

A “system for predicting an atherothrombotic event”, within the meaning of the invention, shall be understood to be any technical arrangement or device suitable for the prediction of atherothrombotic events in a subject. Such a system can be in form of a single laboratory device or can be a combination of one or several laboratory devices, optionally linked with one or several computers. Preferred systems are in a single device.

The present invention relates to a methods of predicting an atherothrombotic event in a human subject, said method comprising the steps of
(i) determining the level of CD154 in said human subject,
(ii) comparing the level determined in step (i) with a reference value, and
(iii) predicting said atherothrombotic event in said human subject from the results of the comparison step (ii).

The invention further relates to a method as defined above, wherein said atherothrombotic event is a myocardial infarction, stroke or mesenteric ischemia.

In further methods of the invention, said atherothrombotic event is death of the subject due to an atherothrombotic event, or said atherothrombotic event is death or hospitalization of said subject due to an atherothrombotic event.

In preferred methods of the invention, said human subject is afflicted with end stage renal disease. Preferably, said human subject is on hemodialysis.

In preferred methods of the invention the prediction of an atherothrombotic event is a determination of the likelihood that an atherothrombotic event occurs in a subject.

One further aspect of the invention relates to methods as defined above, in which said CD154 is soluble CD154 (sCD154).

In preferred methods of the invention said level of CD154 in said human subject is determined from a sample of said human subject. Preferably, this determination is performed ex vivo or in vitro. Preferably, the step of taking a sample is not part of the methods of the invention.

In preferred embodiments of the invention, said level of CD154 in said human subject is determined in blood plasma of said human subject.

In preferred methods of the invention, said level of CD154 in said human subject is determined by chromatography, on a protein chip, on a gene chip, by immunoassay, by homogeneous immunoassay, by heterogenous immunoassay, by competitive immunoassay, by sandwich immunoassay, and/or by mass spectrometry.

In preferred methods of the invention said atherothrombotic event is a fatal atherothrombotic event. In other preferred methods of the invention said atherothrombotic event is a non-fatal atherothrombotic event.

In a preferred embodiment of the invention, the prediction of the atherothrombotic event is specific for atherothrombotic events, i.e. it is not predictive for other cardiovascular events, such as, e.g., sudden cardiac death, sudden cardiac death or hospitalization due to arrhythmia, mortality due to heart failure, mortality or hospitalization due to heart failure, or mortality due to infectious diseases, or mortality or hospitalization due to infectious diseases.

In preferred methods of the invention the reference value is predetermined. Particularly preferred are reference values in the range of 1 to 20 ng/ml, preferably 2 to 15 ng/ml, 3 to 10 ng/ml, 5 to 8 ng/ml, or most preferred 6 to 7 ng/ml (ng protein in ml serum, respectively).

The invention further relates to a system for the prediction of an atherothrombotic event in a human subject, said system comprising
(i) means for the determination of the level of CD154 in a human subject,
(ii) comparison means for comparing the level determined under (i) with a reference value, and
(iii) prediction means for predicting said atherothrombotic event in said human subject from data provided by said comparison means.

The invention further relates to a system as defined above, wherein said means for the determination of the level of CD154 are selected from the group consisting of means for the determination of the level of soluble CD154, means for the determination of the level of soluble CD154 in plasma, means comprising means for performing a chromatography step, a protein chip, a mass spectrometer, and a gene chip.

The invention further relates to a system as defined above, wherein said atherothrombotic event is myocardial infarction, stroke or mesenteric ischemia.

In preferred systems of the invention, said atherothrombotic event is death of the subject due to an atherothrombotic event, or said atherothrombotic events is death or hospitalization of said subject due to an atherothrombotic event.

Preferred systems of the invention predict atherothrombotic events in human subjects which are afflicted with end stage renal disease. Preferably, said human subject is on hemodialysis.

One further aspect of the invention relates to systems as defined above, in which said CD154 is soluble CD154 (sCD154).

In preferred systems of the invention said level of CD154 in said human subject is determined from a sample of said human subject. Preferably, this determination is performed ex vivo or in vitro.

In preferred systems of the invention, said level of CD154 in said human subject is determined in blood plasma of said human subject.

Preferred systems of the invention comprise means for chromatography, a protein chip, a gene chip, means for immunoassay, means for homogeneous immunoassay, means for heterogenous immunoassay, means for competitive immunoassay, means for sandwich immunoassay, and/or a mass spectrometer.

Preferred systems of the invention predict a fatal atherothrombotic event. Other preferred systems of the inventions predict a non-fatal atherothrombotic event.

Preferred systems of the invention perform a specific prediction of the atherothrombotic event, i.e. they do not predict other cardiovascular events, such as, e.g., sudden cardiac death, sudden cardiac death or hospitalization due to arrhythmia, mortality due to heart failure, mortality or hospitalization due to heart failure, or mortality due to infectious diseases, or mortality or hospitalization due to infectious diseases.

In preferred systems of the invention, said reference value is predetermined. Particularly preferred are reference values in the range of 1 to 20 ng/ml, preferably 2 to 15 ng/ml, 3 to 10 ng/ml, 5 to 8 ng/ml, or most preferred 6 to 7 ng/ml (ng protein in ml serum, respectively). Preferred systems of the invention store the predetermined reference value in a memory.

In preferred systems of the invention, the comparison means and the prediction means are in form of computer or a microcontroller. Preferably, the comparison means and the prediction means are in form of a single computer or microcontroller.

In preferred systems of the invention, the means for determining the level of CD154 are linked to the comparison means and/or to the prediction means. Such link can be by a cable or can be wireless. This allows the determination step and the comparison step and the prediction step to be performed at separate locations, e.g. the determination step in a laboratory and the other steps in an office in the vicinity of the laboratory. The link can also be established via the Internet, in which case the office can be at any distance from the laboratory.

Another aspect of the invention relates to systems as described above, further comprising means to perform hemodialysis. In such systems, hemodialysis patients can be treated by hemodialysis while their risk of experiencing a fatal or non-fatal atherothrombotic event is assessed at the same time. Preferably, such a system comprises means to draw samples from the blood passing through the hemodialysis system.

### Example

232 patients were prospectively followed for 52 months. At study entry we documented clinical characteristics and analyzed plasma concentrations of sCD154 and those of conventional risk predictors. Time-point and cause of any hospitalization and death were documented during the entire follow-up. Survival rates were compared with Kaplan-Meier and Cox regression analyses.

122 patients died, 64 patients of cardiovascular disease, including 20 cases of fatal atherothrombotic diseases (myocardial infarction, stroke, mesenteric ischemia). All these 20 cases of fatal atherothrombotic events had high sCD154 plasma levels (cut-off: >6.42 ng/ml) at study entry. The total number of fatal and non-fatal atherothrombotic events was 66. Only 5 atherothrombotic non-fatal events occurred in patients with sCD154<6.42 ng/ml, whereas 61 fatal and non-fatal events were seen in patients with sCD154>6.42 ng/ml (p<0.005). This has been confirmed by Kaplan Meier curves for fatal atherothrombotic events (p=0.0241) and the combined endpoint fatal and non-fatal atherothrombotic events (p=0.0039). Cox regression analysis revealed that high sCD154 is an independent predictor (relative risk: 9.934; CI: 1.338-73.768, p=0.025) for the combined endpoint death or hospitalization due to atherothrombotic events followed by pre-existing coronary heart disease (relative risk: 2.886; CI: 1.426-5.842, p=0.003) and smoking (relative risk: 2.142; CI: 1.120-4.095, p=0.02). Death or hospitalization due to any other reason (arrhythmias, heart failure, infectious diseases and cancer) were not linked to sCD154 plasma concentrations.

This shows that soluble CD154 predicts non-fatal and fatal atherothrombotic events (myocardial infarction, stroke, mesenteric ischemia), but not death and hospitalization due to any other reason, in stable end stage renal disease patients on hemodialysis.

### Study Population

Patients were recruited from two dialysis centers in Berlin, Germany, as described earlier (16;17). The facilities are non-proprietary units from the Kuratorium für Heimdialyse (KfH) (Neu Isenburg, Germany) a unique non-profit facility. All patients were without any actual health problem at study entry. The KIM prescribes a high quality of care corresponding to the U.S. DOQI guidelines and monitors that care by means of computerized data acquisition. The patients are dialyzed at least 3-times weekly in 4-6 h sessions, sufficient to provide a KTN of at least 1.3.

The cohort was recruited in March 2000. Written informed consent was obtained from all participants after protocol approval by the local ethic comity. We excluded patients with malignancies and chronic infections such as osteomyelitis or conditions that might have an impact on the tested serum parameters (see below). The initial documentation included age, gender, underlying renal disease, residual renal function and urinary output, presence of diabetes, hypertension (diagnostic criteria: need for any antihypertensive drug and/or elevated baseline blood pressure), smoking, and presence of coronary heart disease (patients with a history of myocardial infarction, coronary heart disease confirmed by coronary angiograms, or typical stable angina confirmed by use of stress echocardiography or radionuclide studies). Blood samples were taken from all studied patients during March 2000 immediately before dialysis started. The patients were followed up for 52 months starting with the day of blood sampling. We individually evaluated all hospitalizations, deaths and reviewed the records. Time point and reason of each hospitalization and death during the follow-up were documented.

### Biochemical Variables

Albumin, protein, creatinine, cholesterol, triglyceride and D-dimers were measured by standardized autoanalyzer methods (Hitachi 747, Hitachi 911, and STA, respectively, Roche Diagnostics GmbH, Mannheim, Germany). C-reactive protein was measured by standardized methods on an autoanalyzer (Dimenson RxL (DAD, Behring Vertriebs GmbH, Schwalbach, Germany). Troponin T (TNT) was measured with the Elecsys System 2010 (Roche Diagnostics GmbH, Mannheim, Germany). Soluble CD40 ligand was analyzed using a commercial ELISA (Bender Medical Systems, Austria).

### Statistical Analysis

Patients receiving a transplant were censored at the date of the transplantation. Change of treatment modality (initiation of peritoneal dialysis) was also a censoring event.

Categorical data were compared by using the Chi²-test. Continuous variables were assessed with t-tests. Cut-off values of blood parameters (TNT, CrP, cholesterol, fibrinogen, albumin, BNP) for known risk factors were determined as recently described (16;17). For sCD154, we used the lowest quintile of all sCD154 values as cut-off value according to a recent study (15). Using these cut-off values, we analyzed survival with the log-rank tests of Kaplan-Meier curves. Cox regression analyses were performed for the endpoints death and hospitalization due to sudden cardiac death/arrhythmia, heart failure, atherothrombotic events (myocardial infarction, stroke, mesenteric ischemia), infectious diseases and cancer. We included into the Cox regressions sCD154 a priori and factors known to have an influence on the composite endpoint (see above) in end stage renal disease patients. The proportional-hazards assumption, i.e., that the effects of regressors on mortality rates do not vary with time, was assessed by using interaction terms for three follow-up periods. This analysis revealed constant effects of regressors on mortality rates with time. All data were analyzed by using SPSS for Windows (SPSS, Inc., version 12.0, Chicago, Ill.)

### Results:

232 patients on maintenance hemodialysis were followed prospectively for 52 months. The underlying renal diseases are given in Table 1. No patient was lost to follow-up. The mean age of the 119 female and 113 male patients was 65.7113.7 years. 213 patients had hypertension and 78 patients were diabetics.

122 (52.6%) died within 52 month of follow up: 64 patients of cardiovascular disease, including 27 cases of sudden cardiac death, 11 cases of heart failure, 20 cases of fatal atherothrombotic diseases (myocardial infarction, stroke, mesenteric ischemia) and 6 due to fatal bleeding diseases (intracranial hemorrhage, rupture of an aortic aneurysm). Thirty-seven patients died due to infectious diseases and 8 patients following malignancies. The 13 remaining cases of death were caused by various other reasons (accident, hyperkalemia, gastrointestinal bleeding, cessation of dialysis).

Cox proportional hazards analysis of known “classic” factors predicting all-cause death in the study population revealed that troponin T, pre-existing coronary heart disease, CrP and diabetes are the most important all-cause mortality risk factors in our study population (Table 2).

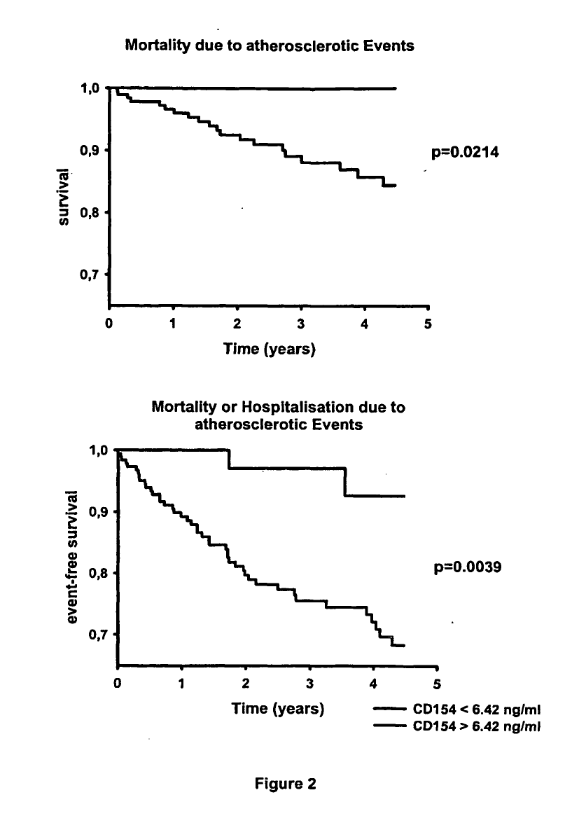

The mean plasma sCD154 concentration was 9.10±3.63 ng/ml. The cut-off for the lowest quintile of sCD154 was 6.42 ng/ml. None of the 47 patients with a sCD154 concentration below the cut-off at study entry died in the following 52 month due to atherothrombotic diseases, whereas 20 patients out of 185 with sCD154≧6.42 ng/ml died due to these diseases during the follow-up (FIG. 2). The mean sCD154 plasma concentration at study entry was also significantly higher (Table 3) in patients dying due to atherothrombotic diseases. The total number of fatal and non-fatal atherothrombotic events (myocardial infarction, strokes and mesenteric ischemia) during the 52 month observation period was 66. Only 5 atherothrombotic non-fatal events occurred in patients with sCD154<6.42 ng/ml, whereas 61 fatal and non-fatal atherothrombotic events were seen in patients with sCD154≧6.42 ng/ml. (p<0.005). Death or hospitalization due to any other reason (arrhythmias, heart failure, infectious diseases and cancer) were not linked to sCD154 plasma concentrations.

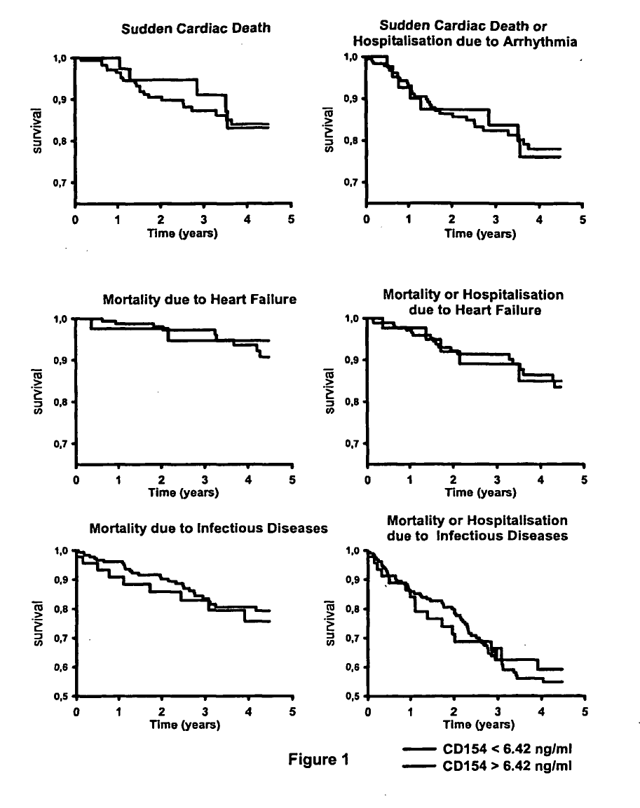

Kaplan Meier curves (FIG. 1) for sudden cardiac death/arrhythmias, heart failure and infectious diseases likewise showed no impact of CD154 on survival and the combined endpoint survival and hospitalization (FIG. 1). Death or the combined endpoint death and hospitalization due to cancer was a relatively rare event (Table 2) and also not related to CD154 plasma levels at study entry (p=0.5461 and p=0.5263, respectively; Kaplan Meier curves not shown).

Kaplan Meier curves for survival and the combined endpoint death/hospitalization (FIG. 2) due to atherothrombotic diseases—on the other hand—showed that CD154 plasma levels at study entry in stable, non-symptomatic ESRD patients predict both fatal and non-fatal atherothrombotic events (myocardial infarction, stroke, mesenteric ischemia).

Cox regression analysis furthermore demonstrated that CD154 is an independent predictor of future death/hospitalization due to atherothrombotic diseases (Table 3). The predictive power of CD154 plasma levels is strikingly specific for atherothrombotic diseases. Death or hospitalization due to any other disease is not related to CD154 plasma levels (FIGS. 1 and 2, Tables 4 and 5). The relative risk of elevated sCD154 for future fatal and non-fatal atherothrombotic events is much higher as the well-established risk predictors smoking or proven pre-existing coronary artery disease at study entry (Table 4).

### DISCUSSION

This prospective study with a follow up of 52 months is the first to demonstrate that soluble CD154 predicts non-fatal and fatal atherothrombotic events (myocardial infarction, stroke, mesenteric ischemia), but not death and hospitalization due to any other reason (sudden cardiac death/arrhythmia, heart failure, infectious diseases and cancer), in asymptomatic and stable end stage renal disease patients on hemodialysis.

The main goal of our study was to analyze the impact of sCD154 on future atherothrombotic events in ESRD patients. However, we were also able to confirm the effects of more-conventional risk factors in predicting all-cause mortality rates among patients with ESRD (Table 2). Cox regression analysis of factors possibly involved in all-cause mortality demonstrated that factors such as preexisting diabetes and coronary artery disease, smoking, elevated CrP levels and increased TNT levels were predictors of all-cause mortality rates for the whole study population. These findings indicate that our study population was representative of patients with ESRD undergoing hemodialysis and that the conclusions of our study are of general applicability (2;3;5;6;8;9;18;19).

So far, sCD154 was mainly recognized as a marker for an acute ongoing atherothrombotic/thrombembolic disease such as acute coronary syndrome (13-15) or acute stroke (20). However, our data indicate that elevated sCD154 is a powerful predictor of non-fatal and fatal atherothrombotic events in apparently asymptomatic ESRD patients on hemodialysis at least for a follow up period of 52 months. Our data are in agreement with a recent nested case-control study among participants in the Women's Health Study, a primary prevention trial evaluating the efficiency of vitamin E and aspirin in 28.263 middle-aged American women (21). This study suggested that sCD154 is associated with increased vascular risk in apparently healthy women. However, it is of note that in ESRD patients we needed only 232 patients to demonstrate an even stronger effect. We suggest that two factors might explain this striking observation: First, the overall markedly enhanced risk for any cardiovascular disease in ESRD patients (6) and second the well-known pro-inflammatory status in ESRD patients (7-9). Both factors may amplify potential effects of the CD40/CD154 system. In this context, it is of note that the blood concentrations of sCD154 in apparently asymptomatic ESRD patients—regardless the outcome—are higher as reported in recent studies in non-ESRD patients even with acute coronary syndromes (13-15;20;21). This is in accordance with results of a study in renal patients published by Schwabe et al. (22).

Our study not only indicates that sCD154 is another strong predictor of further non-fatal and fatal atherothrombotic events. This factor is in addition highly specific to atherothrombotic events. Hospitalization or death due to sudden cardiac death/arrhythmia, heart failure, infectious diseases, and cancer are clearly not related to elevated sCD154 concentrations (FIGS. 1 & 2, and Tables 4 & 5). Moreover, the inflammatory marker C-reactive protein (CrP) predicts all-cause mortality in our study (Table 2) as CrP did in many other studies (7;8;23), but failed to predict mortality and hospitalization due to atherothrombotic events (Table 4). On the other hand subgroup analysis of factors predicting hospitalization and mortality in our study showed that CrP specifically predicts infectious diseases related events (Table 5). Similarly, TNT is a good marker for all-cause mortality (Table 2), but failed to predict atherothrombotic events (Table 4) in our study. Taken together, in multivariate Cox regression analysis, troponin T, CrP, and sCD154 provided independent prognostic information. This is in good agreement with a recent study in patients with acute coronary syndrome (15). Troponins are markers of myocardial necrosis; they are not actively involved in the pathophysiology of atherothrombotic events/coronary syndromes. Troponin T is a sensitive marker for the detection of minor myocardial injury. Our data suggest that ongoing minor myocardial injury might play an important role in the pathogenesis of heart failure at least in ESRD patients (Table 4).

From this study, it follows that CD154 might be a player and not only a marker of future atherothrombotic events caused by atheromatous plaque instability. This concept is supported by convincing animal studies showing that the blockade of CD154 by neutralizing antibodies and also the genetic disruption of CD154 in mice prevent the initiation and progression of atherosclerosis follow up period of 52 months. Our data are in agreement with a recent nested case-control study among participants in the Women's Health Study, a primary prevention trial evaluating the efficiency of vitamin E and aspirin in 28.263 middle-aged American women (21). This study suggested that sCD154 is associated with increased vascular risk in apparently healthy women. However, it is of note that in ESRD patients we needed only 232 patients to demonstrate an even stronger effect. We suggest that two factors might explain this striking observation: First, the overall markedly enhanced risk for any cardiovascular disease in ESRD patients (6) and second the well-known pro-inflammatory status in ESRD patients (7-9). Both factors may amplify potential effects of the CD40/CD154 system. In this context, it is of note that the blood concentrations of sCD154 in apparently asymptomatic ESRD patients—regardless the outcome—are higher as reported in recent studies in non-ESRD patients even with acute coronary syndromes (13-15;20;21). This is in accordance with results of a study in renal patients published by Schwabe et al. (22).

Our study not only indicates that sCD154 is another strong predictor of further non-fatal and fatal atherothrombotic events. This factor is in addition highly specific to atherothrombotic events. Hospitalization or death due to sudden cardiac death/arrhythmia, heart failure, infectious diseases, and cancer are clearly not related to elevated sCD154 concentrations (FIGS. 1 & 2, and Tables 4 & 5). Moreover, the inflammatory marker C-reactive protein (CrP) predicts all-cause mortality in our study (Table 2) as CrP did in many other studies (7;8;23), but failed to predict mortality and hospitalization due to atheiothrombotic events (Table 4). On the other hand subgroup analysis of factors predicting hospitalization and mortality in our study showed that CrP specifically predicts infectious diseases related events (Table 5). Similarly, TNT is a good marker for all-cause mortality (Table 2), but failed to predict atherothrombotic events (Table 4) in our study. Taken together, in multivariate Cox regression analysis, troponin T, CrP, and sCD154 provided independent prognostic information. This is in good agreement with a recent study in patients with acute coronary syndrome (15). Troponins are markers of myocardial necrosis; they are not actively involved in the pathophysiology of atherothrombotic events/coronary syndromes. Troponin T is a sensitive marker for the detection of minor myocardial injury. Our data suggest that ongoing minor myocardial injury might play an important role in the pathogenesis of heart failure at least in ESRD patients (Table 4).

From this study, it follows that CD154 might be a player and not only a marker of future atherothrombotic events caused by atheromatous plaque instability. This concept is supported by convincing animal studies showing that the blockade of CD154 by neutralizing antibodies and also the genetic disruption of CD154 in mice prevent the initiation and progression of atherosclerosis and induces a more stable plaque with fewer inflammatory cells and lipids, and more smooth muscle cells and collagen (24;25).

CD40/CD154 signaling is also important in late atherosclerotic changes, such as lipid core formation and plaque destabilization (25). CD154 is a crucial mediator not only in initial events of atherogenesis but also during the evolution of established atheroma (24;25). In this context it is important to note that statin treatment reduces CD40 and CD154 expression and lowers soluble CD154 (26). Soluble CD154 is shed from stimulated lymphocytes and is actively released after platelet stimulation (27-29). This peptide is pro-inflammatory for endothelial cells and promotes coagulation by inducing expression of tissue factor on monocytes and endothelial cells (30;31).

Soluble CD154 maybe involved in a self-perpetuating feedback loop. Soluble CD154 binds to platelet-bound CD40 and leads to further proteolysis of platelet-associated CD154, and thus further generates sCD154 (32). Soluble CD154 is thus reflecting the activity of the CD40/CD154 system and seems to be also a player in this system.

In conclusion, the present study is the first to demonstrate that soluble CD154 predicts non-fatal and fatal atherothrombotic events (myocardial infarction, stroke, mesenteric ischemia), but not death and hospitalization due to any other reason, in stable end stage renal disease patients on hemodialysis.

TABLE 1
Underlying kidney disease of ESRD patients on maintenance
hemodialysis participating in the study (N = 232); ADPKD:
Autosomal Dominant Polycystic Kidney Disease; CIN: Chronic
Interstitial Nephritis (mainly analgesic nephropathy), HUS:
Hemolytic Uremic Syndrome

| Underlying kidney disease: | N |
|---|---|
| Diabetic nephropathy | 68 |
| Hypertensive Nephrosclerosis | 37 |
| Chronic Glomerulonephritis | 42 |
| ADPKD | 18 |
| CIN | 33 |
| Chronic Pyelonephritis | 2 |
| Congenital Urine Tract Malformations | 5 |
| HUS | 2 |
| Other | 9 |
| Unknown | 16 |

TABLE 2
Cox proportional hazards analysis of factors predicting all-cause death.
Patients were followed for 52 months. Blood was taken at study entry.

| | Relative Risk | 95% CI | p-value |
|---|---|---|---|
| Diabetes | 1.679 | 1.138-2.475 | 0.009 |
| Smoking | 1.662 | 1.125-2.456 | 0.011 |
| Coronary heart disease | 1.803 | 1.204-2.701 | 0.004 |
| Gender Male | 0.696 | 0.455-1.065 | 0.095 |
| Hypertension | 0.442 | 0.248-0.788 | 0.006 |
| Age (per year) | 1.035 | 1.015-1.054 | 0.000 |
| Body Mass Index | 1.013 | 0.973-1.054 | 0.534 |
| Troponin T (>0.039 μg/l) | 3.047 | 1.902-4.880 | 0.000 |
| CrP (>0.730 mg/dl) | 1.690 | 1.085-2.631 | 0.020 |
| Cholesterol (>200 mg/dl) | 1.122 | 0.745-1.690 | 0.582 |
| Fibrinogen (443 mg/dl) | 1.164 | 0.770-1.758 | 0.471 |
| Albumin (<3.70 g/dl) | 0.760 | 0.497-1.164 | 0.207 |
| BNP (>2.5 pg/ml) | 1.374 | 0.936-2.018 | 0.105 |

95% CI: 95% confidence interval

TABLE 3
Baseline clinical data and serum parameters in surviving and non-surviving
hemodialysis. 6 patients died due to bleedings, the remaining 13 died due to other
reasons (accident, hyperkalemia etc.). Fatal atherothrombotic diseases include
death due to myocardial infarction, mesenteric ischemia and stroke. Data are
presented as means ± SD. Chi2-test or unpaired t-test were used when appropriate.

| | | Non-Survivors due to | | | | |
|---|---|---|---|---|---|---|
| | | (N = 103) | | | | |
| | | Sudden | | | | |
| | | cardiac | heart | atherothrombotic | infectious | |
| | Survivors | death | failure | diseases | diseases | Cancer |
| | (N = 110) | (N = 27) | (N = 11) | (N = 20) | (N = 37) | (N = 8) |
| Age at study entry | 59 ± 15 | 71 ± 10** | 71 ± 12** | 69 ± 8** | 73 ± 18** | 69 ± 10* |
| Gender, Female | 54 | 12 | 4 | 8 | 24 | 4 |
| Diabetes mellitus | 21 | 13** | 4 | 11** | 17** | 0 |
| Coronary Heart Disease | 16 | 17** | 1 | 10** | 13** | 1 |
| Hypertension | 106 | 24** | 8** | 17* | 32* | 8 |
| Smoker | 37 | 10 | 5 | 15** | 18 | 3 |
| Time on Dialysis | 4.4 ± 4.0 | 4.5 ± 3.5 | 6.7 ± 7.7 | 5.1 ± 2.9 | 6.4 ± 7.1 | 6.8 ± 4.8 |
| (years) | | | | | | |
| Body mass Index | 25.1 ± 4.2 | 24.6 ± 4.3 | 25.0 ± 5.0 | 28.2 ± 5.7** | 25.2 ± 5.4 | 24.9 ± 4.7 |
| [kg/m²] | | | | | | |
| Serum protein [g/dl] | 6.46 ± 0.4 | 6.53 ± 0.6 | 6.55 ± 0.7 | 6.55 ± 0.4 | 6.50 ± 0.6 | 6.31 ± 0.6 |
| Serum albumin [g/dl] | 3.85 ± 0.3 | 3.81 ± 0.3 | 3.68 ± 0.3 | 3.61 ± 0.3 | 3.56 ± 0.3** | 3.60 ± 0.5 |
| Serum creatinine | 9.73 ± 2.2 | 8.75 ± 2.4 | 8.40 ± 1.5* | 8.56 ± 1.9* | 7.50 ± 1.9** | 8.20 ± 1.7 |
| [mg/dl] | | | | | | |
| CrP [mg/dl] | 0.95 ± 1.1 | 1.10 ± 1.2 | 0.96 ± 1.3 | 2.05 ± 1.4** | 1.74 ± 1.4** | 1.48 ± 1.2 |
| TNT [μg/l] | 0.075 ± 0.28 | 0.097 ± 0.08 | 0.075 ± 0.07 | 0.321 ± 1.01* | 0.260 ± 0.72* | 0.055 ± 0.06 |
| Total Cholesterol | 203.6 ± 51.4 | 220.4 ± 86.9 | 194.5 ± 45.3 | 194.7 ± 53.1 | 207.5 ± 53.2 | 170.3 ± 47.2 |
| [mg/dl] | | | | | | |
| LDL [mg/dl] | 109.6 ± 34.9 | 118.7 ± 49.2 | 112.6 ± 35.5 | 111.3 ± 39.5 | 114.4 ± 33.6 | 90.9 ± 25.8 |
| HDL [mg/dl] | 43.8 ± 13.4 | 40.1 ± 7.4 | 40.4 ± 7.1 | 41.98 ± 12.29 | 40.00 ± 11.01 | 42.3 ± 9.3 |
| Triglycerides [mmol/l] | 186.1 ± 114.5 | 219.3 ± 166.1 | 152.4 ± 63.0 | 157.4 ± 39.4 | 214.2 ± 120.2 | 132.9 ± 69.7 |
| LP(a) [mg/dl] | 30.6 ± 34.2 | 27.3 ± 34.2 | 26.0 ± 31.3 | 18.3 ± 17.5 | 37.5 ± 40.3 | 12.00 ± 11.4 |
| ApoA1 [mg/dl] | 121.6 ± 22.6 | 117.0 ± 13.1 | 114.5 ± 13.8 | 116.0 ± 20.56 | 116.2 ± 16.6 | 117.8 ± 23.6 |
| ApoB [mg/dl] | 104.6 ± 26.9 | 109.0 ± 39.6 | 104.4 ± 26.5 | 103.8 ± 28.5 | 109.4 ± 28.1 | 85.4 ± 19.7* |
| Fibrinogen [mg/dl] | 423.3 ± 97.2 | 457.1 ± 86.9 | 427.3 ± 138.6 | 500.6 ± 103.5** | 480.5 ± 140.9* | 454.6 ± 178.1 |
| D-dimers [mg/dl] | 0.879 ± 1.566 | 1.350 ± 1.836 | 1.30 ± 11.67 | 1.35 ± 1.24 | 1.16 ± 0.96 | 0.773 ± 0.475 |
| BNP [pg/ml] | 9.48 ± 23.6 | 18.23 ± 38.7 | 21.32 ± 29.3 | 14.14 ± 13.8 | 13.22 ± 41.9 | 13.89 ± 22 |
| sCD4OL [ng/ml] | 8.76 ± 3.62 | 10.00 ± 3.47 | 7.21 ± 3.20 | 10.65 ± 2.82* | 9.31 ± 4.38 | 9.41 ± 3.43 |

*p <0.05 compared to survivors;
**p <0.01 compared to survivors.

TABLE 4
Cox proportional hazards analysis of factors predicting death or hospitalization
due to sudden cardiac death, heart failure, atherothrombotic diseases (myocardial
infarction, stroke, mesenteric ischemia). 95% CI: 95% confidence interval

| | Death or Hospitalization due to | | | | | | | | |
|---|---|---|---|---|---|---|---|---|---|
| | Arrhythmia | | | Heart failure | | | Atherothrombotic events | | |
| | Relative | | | Relative | | | Relative | | |
| | Risk | 95% CI | p-value | Risk | 95% CI | p-value | Risk | 95% CI | p-value |
| sCD40L | 1.103 | 0.457- | 0.828 | 0.816 | 0.283- | 0.707 | 9.934 | 1.338- | 0.025 |
| (>6.42 ng/ml) | | 2.664 | | | 2.357 | | | 73.768 | |
| Diabetes | 1.780 | 0.914- | 0.090 | 1.917 | 0.783- | 0.154 | 0.798 | 0.412- | 0.502 |
| | | 3.466 | | | 4.694 | | | 1.544 | |
| Smoking | 0.978 | 0.474- | 0.953 | 0.789 | 0.317- | 0.610 | 2.142 | 1.120- | 0.021 |
| | | 2.018 | | | 1.964 | | | 4.095 | |
| Coronary | 2.776 | 1.392- | 0.004 | 1.091 | 0.397- | 0.866 | 2.886 | 1.426- | 0.003 |
| Heart Disease | | 5.535 | | | 2.999 | | | 5.842 | |
| Male Gender | 1.330 | 0.632- | 0.453 | 0.913 | 0.356- | 0.850 | 0.648 | 0.315- | 0.239 |
| | | 2.798 | | | 2.342 | | | 1.334 | |
| Hypertension | 0.337 | 0.139- | 0.016 | 0.369 | 0.104- | 0.125 | 0.590 | 0.235- | 0.261 |
| | | 0.815 | | | 1.318 | | | 1.481 | |
| Age (per year) | 1.038 | 1.006- | 0.018 | 1.009 | 0.975- | 0.609 | 1.028 | 0.997- | 0.075 |
| | | 1.071 | | | 1.045 | | | 1.061 | |
| Body Mass | 0.966 | 0.891- | 0.394 | 1.107 | 1.014- | 0.024 | 1.070 | 1.007- | 0.029 |
| Index | | 1.046 | | | 1.208 | | | 1.136 | |
| Troponin T | 1.342 | 0.626- | 0.449 | 2.820 | 1.032- | 0.043 | 1.508 | 0.749- | 0.250 |
| (>0.039 μg/l) | | 2.877 | | | 7.702 | | | 3.040 | |
| CrP | 0.815 | 0.363- | 0.621 | 1.047 | 0.384- | 0.929 | 1.536 | 0.704- | 0.281 |
| (>0.730 mg/dl) | | 1.831 | | | 2.854 | | | 3.349 | |
| Cholesterol | 0.872 | 0.422- | 0.712 | 1.533 | 0.603- | 0.370 | 1.453 | 0.740- | 0.278 |
| (>200 mg/dl) | | 1.802 | | | 3.896 | | | 2.854 | |
| Fibrinogen | 1.244 | 0.599- | 0.558 | 0.445 | 0.163- | 0.114 | 0.734 | 0.358- | 0.398 |
| (>443 mg/dl) | | 2.585 | | | 1.213 | | | 1.504 | |
| Albumin | 1.104 | 0.528- | 0.792 | 1.035 | 0.378- | 0.946 | 1.007 | 0.508- | 0.985 |
| (<3.7 g/dl) | | 2.310 | | | 2.835 | | | 1.995 | |
| BNP | 1.039 | 0.526- | 0.913 | 1.011 | 0.409- | 0.981 | 2.000 | 1.055- | 0.034 |
| (>2.5 pg/ml ) | | 2.050 | | | 2.496 | | | 3.790 | |

TABLE 5
Cox proportional hazards analysis of factors predicting death or hospitalization
due to infectious diseases or cancer. (95% CI: 95% confidence interval)

| | Death or Hospitalization due to | | | | | |
|---|---|---|---|---|---|---|
| | Infectious Diseases | | | Cancer | | |
| | Relative | | | Relative | | |
| | Risk | 95% CI | p-value | Risk | 95% CI | p-value |
| sCD40L | 0.779 | 0.424-1.429 | 0.420 | 1.440 | 0.390-5.309 | 0.584 |
| (>6.42 ng/ml ) | | | | | | |
| Diabetes | 1.239 | 0.745-2.059 | 0.409 | 0.270 | 0.059-1.238 | 0.092 |
| Smoking | 1.285 | 0.784-2.104 | 0.320 | 0.528 | 0.163-1.715 | 0.288 |
| Coronary Heart Disease | 0.810 | 0.460-1.425 | 0.464 | 0.663 | 0.142-3.086 | 0.600 |
| Male Gender | 0.864 | 0.504-1.483 | 0.596 | 0.444 | 0.140-1.406 | 0.167 |
| Hypertension | 0.891 | 0.377-2.103 | 0.792 | 1.532 | 0.193-12.151 | 0.686 |
| Age (per year) | 1.032 | 1.010-1.055 | 0.004 | 1.047 | 1.006-1.091 | 0.026 |
| Body Mass Index | 1.006 | 0.956-1.058 | 0.814 | 1.018 | 0.907-1.142 | 0.765 |
| Troponin T | 1.658 | 0.979-2.806 | 0.060 | 0.946 | 0.338-2.649 | 0.916 |
| (>0.039 μg/l) | | | | | | |
| CrP | 2.089 | 1.195-3.651 | 0.010 | 1.706 | 0.548-5.314 | 0.357 |
| (>0.730 mg/dl) | | | | | | |
| Cholesterol | 1.493 | 0.889-2.510 | 0.130 | 0.573 | 0.201-1.636 | 0.298 |
| (>200 mg/dl) | | | | | | |
| Fibrinogen | 1.010 | 0.597-1.708 | 0.971 | 0.901 | 0.303-2.677 | 0.851 |
| (>443 mg/d) | | | | | | |
| Albumin | 0.609 | 0.358-1.035 | 0.067 | 1.544 | 0.489-4.878 | 0.459 |
| (<3.70 g/dl) | | | | | | |
| BNP | 0.806 | 0.485-1.340 | 0.406 | 1.172 | 0.428-3.208 | 0.757 |
| (>2.5 pg/ml ) | | | | | | |

### REFERENCE LIST

(1) Brunner F P, Selwood N H. Profile of patients on RRT in Europe and death rates due to major causes of death groups. The EDTA Registration Committee. Kidney Int Suppl 1992; 38:S4-15.
(2) Port F K. Morbidity and mortality in dialysis patients. Kidney Int 1994; 46(6):1728-1737.
(3) Excerpts from United States Renal Data System 1999 Annual Data Report. Am J Kidney Dis 1999; 34(2 Suppl 1):S1-176.
(4) Byrne C, Vernon P, Cohen J J. Effect of age and diagnosis on survival of older patients beginning chronic dialysis. JAMA 1994; 271(1):34-36.
(5) Morbidity and mortality of dialysis. NIH Consens Statement 1993; 11(2):1-33.
(6) Foley R N, Parfrey P S, Samak M J. Clinical epidemiology of cardiovascular disease in chronic renal disease. Am J Kidney Dis 1998; 32(5 Suppl 3):S112-S119.
(7) Shlipak M G, Fried L F, Crump C, Bleyer A J, Manolio T A, Tracy R P et al. Elevations of inflammatory and procoagulant biomarkers in elderly persons with renal insufficiency. Circulation 2003; 107(1):87-92.
(8) Tripepi G, Mallamaci F, Zoccali C. Inflammation markers, adhesion molecules, and all-cause and cardiovascular mortality in patients with ESRD: searching for the best risk marker by multivariate modeling. J Am Soc Nephrol 2005; 16 Suppl 1:S83-S88.
(9) Liu Y, Berthier-Schaad Y, Fallin M D, Fink N E, Tracy R P, Klag M J et al. IL-6 haplotypes, inflammation, and risk for cardiovascular disease in a multiethnic dialysis cohort. J Am Soc Nephrol 2006; 17(3):863-870.
(10) Granger D N, Vowinkel T, Petnehazy T. Modulation of the inflammatory response in cardiovascular disease. Hypertension 2004; 43(5):924-931.
(11) Schonbeck U, Libby P. CD40 signaling and plaque instability. Circ Res 2001; 89(12):1092-1103.
(12) Andre P, Prasad K S, Denis C V, He M, Papalia J M, Hynes R O et al. CD40L stabilizes arterial thrombi by a beta3 integrin—dependent mechanism. Nat Med 2002; 8(3):247-252.
(13) Aukrust P, Muller F, Ueland T, Berget T, Aaser E, Brunsvig A et al. Enhanced levels of soluble and membrane-bound CD40 ligand in patients with unstable angina. Possible reflection of T lymphocyte and platelet involvement in the pathogenesis of acute coronary syndromes. Circulation 1999; 100(6):614-620.
(14) Nannizzi-Alaimo L, Rubenstein M H, Alves V L, Leong G Y, Phillips D R, Gold H K. Cardiopulmonary bypass induces release of soluble CD40 ligand. Circulation 2002; 105(24):2849-2854.
(15) Heeschen C, Dimmeler S, Hamm C W, van den Brand M J, Boersma E, Zeiher A M et al. Soluble CD40 ligand in acute coronary syndromes. N Engl J Med 2003; 348(12):1104-1111.
(16) Hocher B, Ziebig R, Alternann C, Krause R, Asmus G, Richter C M et al. Different impact of biomarkers as mortality predictors among diabetic and nondiabetic patients undergoing hemodialysis. J Am Soc Nephrol 2003; 14(9):2329-2337.
(17) Hocher B, Ziebig R, Krause R, Asmus G, Neumayer H H, Liefeldt L et al. Relaxin is an independent risk factor predicting death in male patients with end-stage kidney disease. Circulation 2004; 109(19):2266-2268.
(18) Herzog C A, Ma J Z, Collins A J. Comparative survival of dialysis patients in the United States after coronary angioplasty, coronary artery stenting, and coronary artery bypass surgery and impact of diabetes. Circulation 2002; 106(17):2207-2211.
(19) Dierkes J, Domrose U, Westphal S, Ambrosch A, Bosselmann H P, Neumann K H et al. Cardiac troponin T predicts mortality in patients with end-stage renal disease. Circulation 2000; 102(16):1964-1969.
(20) Garlichs C D, Kozina S, Fateh-Moghadam S, Handschu R, Tomandl B, Stumpf C et al. Upregulation of CD40-CD40 ligand (CD154) in patients with acute cerebral ischemia. Stroke 2003; 34(6):1412-1418.
(21) Schonbeck U, Varo N, Libby P, Buring J, Ridker P M. Soluble CD40L and cardiovascular risk in women. Circulation 2001; 104(19):2266-2268.
(22) Schwabe R F, Engelmann H, Hess S, Fricke H. Soluble CD40 in the serum of healthy donors, patients with chronic renal failure, haemodialysis and chronic ambulatory peritoneal dialysis (CAPD) patients. Clin Exp Immunol 1999; 117(1):153-158.
(23) Wanner C, Metzger T. C-reactive protein a marker for all-cause and cardiovascular mortality in haemodialysis patients. Nephrol Dial Transplant 2002; 17 Suppl 8:29-32.
(24) Lutgens E, Gorelik L, Daemen M J, de Muinck E D, Grewal I S, Koteliansky V E et al. Requirement for CD154 in the progression of atherosclerosis. Nat Med 1999; 5(11):1313-1316.
(25) Lutgens E, Cleutjens K B, Heeneman S, Koteliansky V E, Burkly L C, Daemen M J. Both early and delayed anti-CD40L antibody treatment induces a stable plaque phenotype. Proc Natl Acad Sci USA 2000; 97(13):7464-7469.
(26) Semb A G, van Wissen S, Ueland T, Smilde T, Waehre T, Tripp M D et al. Raised serum levels of soluble CD40 ligand in patients with familial hypercholesterolemia: downregulatory effect of statin therapy. J Am Coll Cardiol 2003; 41(2):275-279.
(27) Graf D, Muller S, Korthauer U, van Kooten C, Weise C, Kroczek R A. A soluble form of TRAP (CD40 ligand) is rapidly released after T cell activation. Eur J Immunol 1995; 25(6):1749-1754.
(28) Lee Y, Lee W H, Lee S C, Aim K J, Choi Y H, Park SW et al. CD40L activation in circulating platelets in patients with acute coronary syndrome. Cardiology 1999; 92(1):11-16.
(29) Henn V, Steinbach S, Buchner K, Presek P, Kroczek RA. The inflammatory action of CD40 ligand (CD154) expressed on activated human platelets is temporally limited by coexpressed CD40. Blood 2001; 98(4):1047-1054.
(30) Urbich C, Mallat Z, Tedgui A, Clauss M, Zeiher A M, Dimmeler S. Upregulation of TRAF-3 by shear stress blocks CD40-mediated endothelial activation. J Clin Invest 2001; 108(10):1451-1458.
(31) Scarborough R M, Naughton M A, Teng W, Rose J W, Phillips D R, Nannizzi L et al. Design of potent and specific integrin antagonists. Peptide antagonists with high specificity for glycoprotein IIb-IIIa. J Biol Chem 1993; 268(2):1066-1073.
(32) Anand S X, Viles-Gonzalez I F, Badimon J J, Cavusoglu E, Marmur J D. Membrane-associated CD40L and sCD40L in atherothrombotic disease. Thromb Haemost 2003; 90(3):377-384.

1. Method for predicting an atherothrombotic event in a human subject, comprising the steps of
(i) determining the level of CD154 in said human subject,
(ii) comparing the level determined under (i) with a reference value, and
(iii) predicting said atherothrombotic event in said human subject from the results of said comparison step under (ii).

2. Method of claim 1, wherein said atherothrombotic event is myocardial infarction, stroke or mesenteric ischemia.

3. Method of any of the preceding claims, wherein said human subject is afflicted with end stage renal disease (ESRD).

4. Method of claim 3, wherein said human subject is asymptomatic.

5. Method of any of the preceding claims, wherein said human subject is on hemodialysis.

6. Method of any of the preceding claims, wherein said CD154 is soluble CD154.

7. Method of any of the preceding claims, wherein said level of CD154 in said human subject is determined from a sample of said human subject.

8. Method of any of the preceding claims, wherein said level of CD154 in said human subject is determined in blood plasma.

9. Method of any of the preceding claims, wherein said level of CD154 in said human subject is determined by chromatography, and/or on a protein chip, and/or by a mass spectrometer, and/or on a gene chip, and/or by immunoassay, and/or by a homogeneous immunoassay, and/or by a heterogenous immunoassay, and/or by a competitive immunoassay, and/or by a sandwich immunoassay, and/or by mass spectrometry.

10. Method of any of the preceding claims, wherein said prediction is independent from other cardiovascular events.

11. Method of any of the preceding claims, wherein said reference value is predetermined.

12. Method of any of the preceding claims, wherein said reference value is in the range of 1 to 20 ng Protein/ml sample.

13. System for the prediction of an atherothrombotic event in a human subject, said system comprising
(i) means for the determination of the level of CD154 in a human subject,
(ii) comparison means for comparing the level determined under (i) with a reference value, and
(iii) prediction means for predicting said atherothrombotic event in said human subject from data provided by said comparison means.

14. A system of claim 13, further comprising means to perform hemodialysis.
